(19) United States
(12) Patent Application Publication
(10) Pub. No.: US 2016/0106599 A1
(43) Pub. Date: Apr. 21, 2016

(54) FIBER-WEAVING HYGIENE ITEM AND THE FABRICATING METHOD THEREOF

(71) Applicant: National Tsing Hua University, Hsinchu (TW)

(72) Inventors: Chung-Yao YANG, Hsinchu (TW); Szu-Ting LIN, Hsinchu (TW); Susan Madeline OSCAR, Hsinchu (TW); Hsin-Hung LIN, Hsinchu (TW); Ting-Yu LIN, Hsinchu (TW); Chao-Min CHENG, Hsinchu (TW)

(21) Appl. No.: 14/656,931

(22) Filed: Mar. 13, 2015

(30) Foreign Application Priority Data
Oct. 16, 2014 (TW) 103135897

(51) Int. Cl. A61F 13/42; A61F 13/15; G01N 21/78

(52) U.S. Cl. CPC A61F 13/42; G01N 21/78; A61F 13/15585; A61F 13/15617; A61F 2013/427

(57) ABSTRACT

The present invention provides a fiber-weaving hygiene item and the fabricating method thereof. The hygiene item comprises a substrate, a fabric-base fiber, and a sensing matter. The fabricating method comprises the following steps: preparing a substrate, forming a plurality of penetrating holds on the substrate, preparing a fabric-base fiber and interlacing the fabric-base fiber on a first surface and a second surface of the substrate, and disposing a sensing matter on the fabric-base fiber, wherein the hydrophilicity of the fabric-base fiber is higher than that of the substrate. The present invention applies the characteristic of the higher hydrophilicity of the fabric-base fiber for luring a matter under test toward the fabric-base fiber. When the matter under test is contacted with the fabric-base fiber, the sensing matter can change its color for bio-examination. Using the present invention, multiple bio-examinations can be done for examining the matter under test.

### BACKGROUND OF THE INVENTION

1. Field of the Invention

The present invention discloses a hygiene item and the fabricating method thereof; and more particularly, the present invention discloses a fiber-weaving hygiene item and the fabricating method thereof.

2. Description of the Prior Art

The hygiene item having liquid examination function for examining each chemical matter in the body-fluids in the prior art is mostly manufactured through dipping papers into the liquid sensing material (e.g. enzymes or oxidases) for allowing the liquid sensing material to be absorbed uniformly by the paper. When the test paper in the prior art is applied, the sensing material in the test paper reacts with the predetermined matter in the testing liquid for allowing the color of the sensing material in the test paper to be changed, which further allows the user to know whether the predetermined matter is comprised in the testing liquid or not for achieving the goal of examination. Furthermore, there are some other applications such as dropping or spreading the sensing material on the surface of the test sheets (individual hygiene products like diapers, sanitary napkins and so on) for examining the chemical matters comprised in the body-fluids.

Take the U.S. Pat. No. 6,203,496 for example. This patent discloses a diaper, composed of a permeable layer, an absorption layer, and an impermeable layer. A chemical reagent is set for sensing the matter comprised in the urine by color change. The chemical reagent is directly dropped in the absorption layer to react with chemical matters comprised in the urine. When there is only a single reagent comprised, the effect of the application is less influenced. But, if the producer wants to drop various kinds of reagents in the absorption layer of the diaper at the same time, different kinds of reagents will be mixed due to capillarity, and then the reagents will lose efficacy.

Additionally, as shown in the Taiwan patent publication No. M456182, the patent discloses a method for examining the pH value of the urine by directly spreading the acid-base indicator on the inner surface of the impermeable outer layer of the diaper. Similarly, this patent utilizes the absorption layer to absorb the reagent for examination, and induces the same problem which can not support different kinds of reagents.

More particularly, nowadays, there is no art can effectively produce a hygiene item having various kinds of sensing materials with low cost. Therefore, the application is limited and fails to be promoted and applied.

The statement mentioned above is the current state of art of the hygiene item which has the function of chemical examination.

### SUMMARY OF THE INVENTION

To solve the problem mentioned above, through testing and researching repeatedly, the inventor provides a hygiene item with simple fabrication, low cost, and the ability of sensing various kinds of chemical matters at the same time. The structure and the fabricating method of the hygiene item are disclosed in the present invention.

The major category of the present invention is to provide a fiber-weaving hygiene item, used for performing a bio-examination to a matter under test, mainly comprising: a substrate, a fabric-base fiber, and a sensing matter. The substrate has a first surface, a second surface, and a plurality of penetrating holds. The fabric-base fiber is interlaced on the first surface and the second surface through the plurality of penetrating holds. The sensing matter is disposed on the fabric-base fiber on the first surface, wherein the hydrophilicity of the fabric-base fiber is higher than the hydrophilicity of the substrate. When the matter under test is contacted with the first surface of the substrate, the color of the sensing matter of the fabric-base fiber will be changed for bio-examination.

Wherein, the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously. The nitrite sensing reagent comprises 50 mM sulfanilamide, 330 mM citric acid, and 10 mM N-(1-naphthyl) ethylenediamine; the glucose sensing reagent comprises 75 U/mL glucose oxidase, 15 U/mL horseradish peroxidase, and 0.6 M potassium iodide; the bovine serum albumin (BSA) sensing reagent comprises 250 mM citric acid and 3.9 mM tetrabromophenol blue; the bilirubin sensing reagent comprises 4.9 mM sodium nitrite, 145 mM sulfanilic acid, and 104 mM hydrochloric acid; the urobilinogen sensing reagent comprises 0.1 M 4-dimethylaminobenzaldehyde and 2.8 M hydrochloric acid; the pH sensing reagent comprises 0.02% methyl red and 0.25% bromothymol Blue; and the ketone body sensing reagent comprises 3% sodium nitroprusside and 0.2 M glycine.

Another category of the present invention is to provide a fabricating method for the fiber-weaving hygiene item, comprising the following steps: (S1) preparing a substrate, wherein the substrate has a first surface and a corresponding second surface; (S2) forming a plurality of penetrating holds on the substrate; (S3) preparing a fabric-base fiber and interlacing the fabric-base fiber on the first surface and the second surface through the plurality of penetrating holds, and (S4) disposing a sensing matter on the fabric-base fiber on the first surface, wherein the hydrophilicity of the fabric-base fiber is higher than the hydrophilicity of the substrate. When a matter under test is contacted with the first surface of the substrate, the color of the sensing matter of the fabric-base fiber will be changed for bio-examination, wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, and the sensing reagents can be used respectively or simultaneously.

Another category of the present invention is to provide a fiber-weaving hygiene item, used for performing a bio-examination to a matter under test, mainly comprising a fiber layer and a sensing matter. The fiber layer comprises a hydrophilic fiber and a hydrophobic fiber, and the hydrophilic fiber and the hydrophobic fiber are interlaced to form the fiber layer. Also, the sensing matter is disposed on the hydrophilic fiber. When a matter under test is contacted with the hydrophilic fiber, the sensing matter of the hydrophilic fiber will change color for bio-examination.

Wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously.

Another category of the present invention is to provide a fabricating method for the fiber-weaving hygiene item, comprising the following steps: (K1) preparing a hydrophilic fiber and a hydrophobic fiber; (K2) interlacing the hydrophilic fiber and the hydrophobic fiber to form a fiber layer, and (K3) disposing a sensing matter on the hydrophilic fiber, wherein when a matter under test is contacted with the hydrophilic fiber, the sensing matter of the hydrophilic fiber will change color for bio-examination.

Wherein the sensing matter comprises a nitrite sensing reagent, a glucose sensing reagent, a bovine serum albumin (BSA) sensing reagent, a pH sensing reagent, bilirubin sensing reagent, an urobilinogen sensing reagent, and a ketone body sensing reagent, wherein the sensing reagents can be used respectively or simultaneously.

To compare with the prior art, the present invention provides a fiber-weaving hygiene item, which interlaces the hydrophilic fiber and the hydrophobic fiber, or interlaces the hydrophilic fiber on the hydrophobic substrate for allowing the hydrophilic fiber to he distributed on the hydrophobic material, and disposes the sensing matter on the hydrophilic fiber for bio-examination of the matter under test. By means of the present invention, one or a plurality of sensing matters can be disposed on the hydrophilic fiber for performing the bio-examination of one kind or above to the matter under test, to solve the problem that the hygiene item can not inspect a variety of reagents in the prior art.

The advantages and spirits of the invention may be understood by the following recitations together with the appended drawings.

### BRIEF DESCRIPTION OF THE APPENDED DRAWINGS

Some of the embodiments will be described in detail, with reference to the following figures, wherein like designations denote like members, wherein:

FIG. 1 shows an explosion diagram of the hygiene item according to an embodiment of the present invention.

FIG. 1A shows a schematic diagram of fiber threading of the hygiene item according to an embodiment of the present invention.

FIG. 2 shows a step flow block diagram for fabricating the hygiene item according to an embodiment of the present invention.

FIG. 2A shows a step flow block diagram for fabricating the hygiene item according to an embodiment of the present invention.

FIG. 3 and FIG. 4 show the explosion diagrams of the hygiene item according to another embodiment of the present invention.

FIG. 3A and FIG. 3B show the schematic diagrams of the arrangement of the hydrophilic fiber comprising the sensing matter of the hygiene item according to an embodiment of the present invention.

FIG. 5 shows a step flow block diagram for fabricating the hygiene item according to another embodiment of the present invention.

### DETAILED DESCRIPTION OF THE INVENTION

A detailed description of the hereinafter described embodiments of the disclosed apparatus and method is presented herein by way of exemplifications with reference to the Figures. Although certain embodiments are shown and described in detail, it should be understood that various changes and modifications may be made without departing from the scope of the appended claims The scope of the present invention will in no way be limited to the number of constituting components, the materials thereof, the shapes thereof, the relative arrangement thereof, etc., and are disclosed simply as an example of embodiments of the present invention.

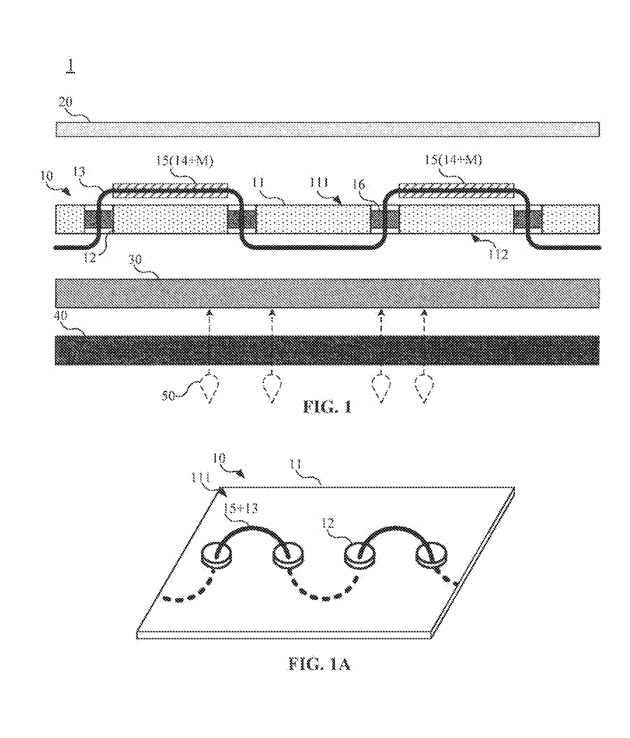

Please refer to FIG. 1 and FIG. 1A. FIG. 1 shows an explosion diagram of the hygiene item according to an embodiment of the present invention and FIG. 1A shows a schematic diagram of fiber threading of the hygiene item according to an embodiment of the present invention. In this embodiment, the present invention provides a fiber-weaving hygiene item, wherein the fiber-weaving hygiene item can be a baby diaper, an adult diaper, or a sanitary napkin. The present invention can be a disposable hygiene item, which can be contacted with the body-fluids of the user, such as sweat, saliva, urine, menstruation, and other fluids outputted from the human body, for bio-examination. The hygiene item 1 provided by the present invention comprises a substrate 11, a plurality of penetrating holds 12, a fabric-base fiber 13, and a sensing matter 14.

Wherein the substrate 11 has a first surface 111 and a second surface 112, and the substrate 11 can be a hydrophobic material or a gel film. The plurality of penetrating holds 12 can be formed from processing the substrate 11 by laser cutting, punching, and drilling The fabric-base fiber 13 is a hydrophilic material. The hydrophilicity of the fabric-base fiber 13 is higher than the hydrophilicity of the substrate 11. The sensing matter 14 comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent.

As shown in FIG. 1 and FIG. 1A, the fabric-base fiber 13 is interlaced on the first surface 111 and the second surface 112 of the substrate 11 through the plurality of penetrating holds 12. For example, distribute the fabric-base fiber 13 on the first surface 111 and the second surface 112 by the method of weaving. Then, dispose a sensing matter 14 on the fabric-base fiber 13 on the first surface 111. When the matter under test 50, such as sweat, saliva, urine, menstruation, or other fluids outputted from the human body, is contacted with the first surface 111, the color of the sensing matter 14 of the fabric-base fiber 13 will be changed for bio-examination. Wherein the hygiene item 1 of the present invention further comprises a waterproof sealant 16, used to be filled in the plurality of penetrating holds 12 for fixing the fabric-base fiber 13 on the first surface 111 and the second surface 112.

Additionally, in order to avoid the sensing matter 14 to contact with air for a long time and then to influence the valid period of the sensing matter 14, the sensing matter 14 can be covered by gel M to form a mixed slurry 15. The mixed slurry 15 mentioned herein is used to explain the co-existence state of the sensing matter 14 and the gel M. When necessary, the term of mixed slurry also can be known as the state after solidification. The valid period of the sensing matter 14 can be prolonged by covering the sensing matter 14 with gel M to reduce the contact area of the sensing matter 14 and air. In practical application, the matter under test 50 can be melted and then pass through the gel M to react with the sensing matter 14, which allows the color of the sensing matter 14 to be changed for bio-examination.

Wherein as shown in FIG. 1A, there is a distance between each fabric-base fiber 13 distributed on the first surface 111, so the sensing matter 14 disposed on the fabric-base fiber 13 can comprise but not be limited to one of the sensing reagents, including a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent. In practical application, the seven sensing reagents, or other sensing reagents except the seven sensing reagents mentioned above, can be disposed on the different position of the fabric-base fiber 13 on the first surface 111 at the same time. The two mixed slurries 15 shown in FIG. 1 can be mixed with different sensing matter 14 respectively for different types of bio-examination.

In this embodiment, the structure mentioned above can form a sensing layer 10 first, and then combines the sensing layer 10 with a gel film 20, an absorption layer 30, and a hydrophobic layer 40 to form the hygiene item 1.

As shown in FIG. 1, in the hygiene item 1 of the present invention, the sensing layer 10 is disposed between the gel film 20 and the hydrophobic layer 40, and the hydrophobic layer 40 is disposed between the sensing layer 10 and the absorption layer 30. The absorption layer 30 is contacted with the skin of the user and adapted to receive the matter under test 50 outputted from the user. The matter under test 50 is transmitted to the sensing layer 10 through the hydrophobic layer 40. The gel film 20 is facing to the external environment. The gel film 20 is a transparent gel film or a translucent gel film for allowing the user to observe the mixed slurry 15 directly.

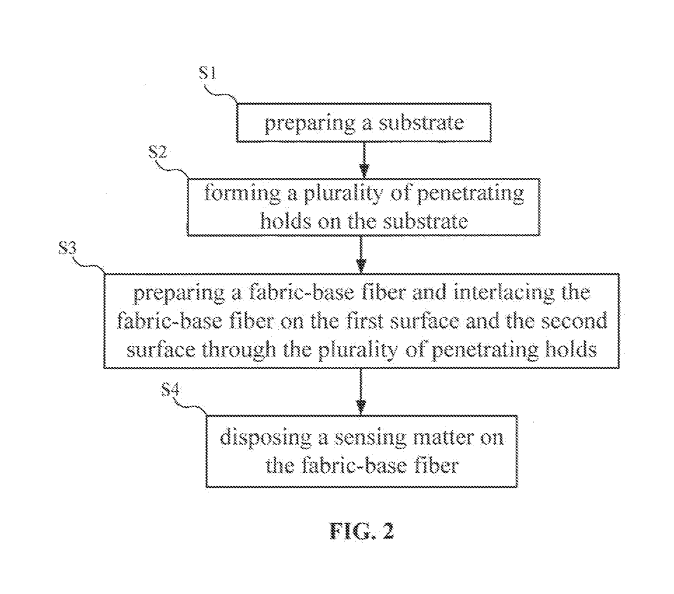

Please refer to FIG. 2 and FIG. 2A. FIG. 2 shows a step flow block diagram for fabricating the hygiene item according to an embodiment of the present invention, and FIG. 2A shows a step flow block diagram for fabricating the hygiene item according to an embodiment of the present invention. As shown in FIG. 2, another category of the present invention is to provide a fabricating method for fiber-weaving hygiene item 1, comprising the following steps: (S1) preparing a substrate, (S2) forming a plurality of penetrating holds on the substrate, (S3) preparing a fabric-base fiber and interlacing the fabric-base fiber on the first surface and the second surface through the plurality of penetrating holds, and (S4) disposing a sensing matter on the fabric-base fiber on the first surface.

In step (S1), prepare a substrate 11, wherein the substrate 11 has a first surface 111 and a corresponding second surface 112. Then perform the step (S2) on the substrate 11 by means of the processing method of laser cutting, punching, and drilling, wherein the step (S2) is to form a plurality of penetrating holds 12 on the substrate 11. Next, perform step (S3): preparing a fabric-base fiber 13 and interlacing the fabric-base fiber 13 on the first surface 111 and the second surface 112 through the plurality of penetrating holds 12. For example, distribute the fabric-base fiber 13 on the first surface 111 and the second surface 112 by the method of weaving. After that, perform step (S4): disposing a sensing matter 14 on the fabric-base fiber 13 on the first surface 111. When a matter under test 50 is transmitted through the second surface 112 of the substrate 11 and contacted with the first surface 111 of the substrate 11, the color of the sensing matter 14 of the fabric-base fiber 13 will be changed for bio-examination.

Through the steps mentioned above, the sensing layer 10 can be formed first, and then the sensing layer 10 will be combined with a gel film 20, an absorption layer 30, and a hydrophobic layer 40 to form the hygiene item 1. The relative position of the sensing layer 10, the gel film 20, the absorption layer 30, and the hydrophobic layer 40 can be referred to FIG. 1 and the relative statement mentioned above.

Wherein the substrate 11 can be a hydrophobic material or a gel film, and the fabric-base fiber 13 can be a hydrophilic material. The sensing matter 14 comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent. The hydrophilicity of the fabric-base fiber 13 is higher than the hydrophilicity of the substrate 11. Besides, in order to avoid the sensing matter 14 to contact with air for a long time and then to influence the valid period of the sensing matter 14, the sensing matter 14 can be covered by gel M to form a mixed slurry 15. In practical application, the matter under test 50 can be melted and then passes through the gel M to react with the sensing matter 14, which allows the color of the sensing matter 14 to be changed for examination.

Please refer to FIG. 2A. The present invention provides a fabricating method for fiber-weaving hygiene item, wherein between the step (S3) and (S4), the step (S31) is further comprised of filling a waterproof sealant 16 in the plurality of penetrating holds 12 for fixing the fabric-base fiber 13 on the first surface 111 and the second surface 112.

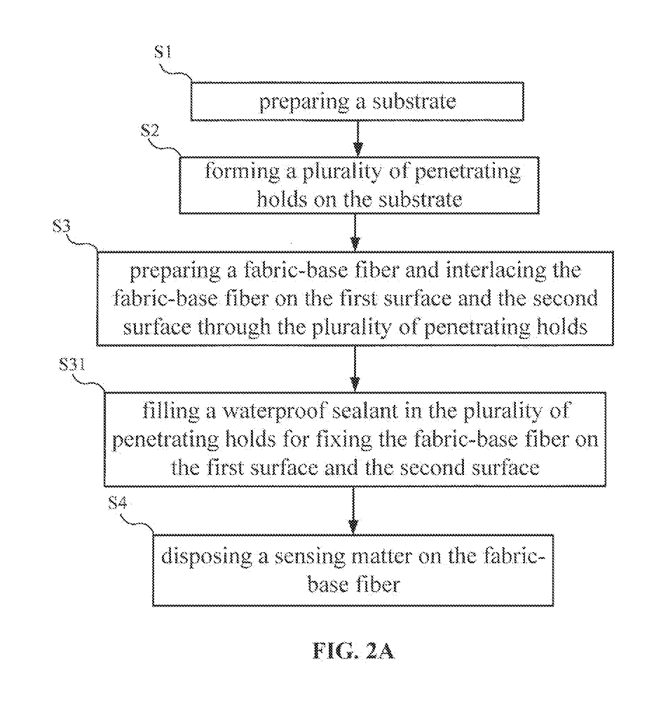

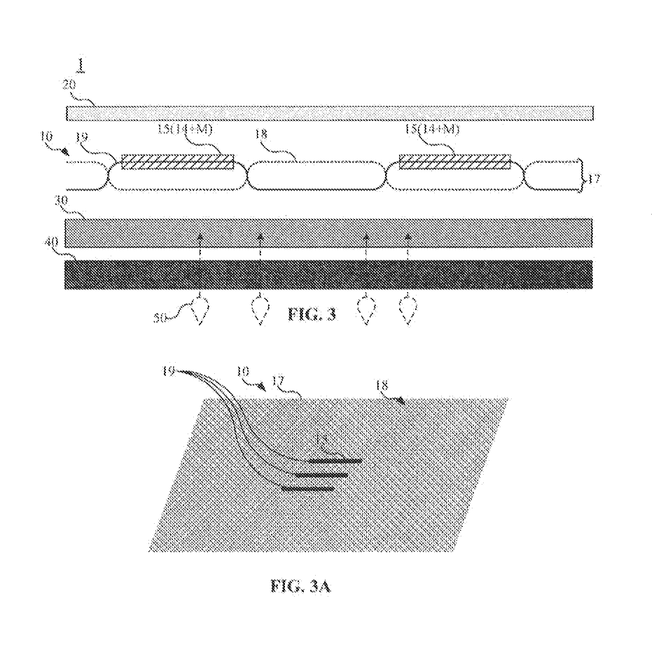

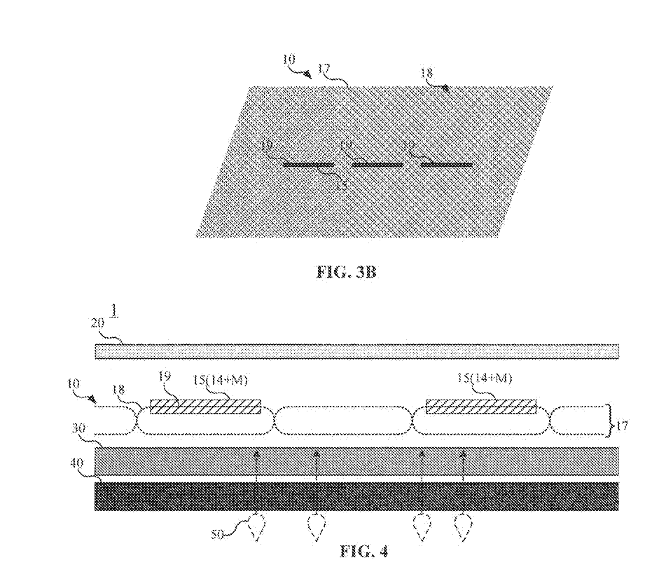

Please refer to FIG. 3 to FIG. 4. FIG. 3 and FIG. 4 show the explosion diagrams of the hygiene item according to another embodiment of the present invention, and FIG. 3A and FIG. 3B shows the schematic diagrams of the arrangement of the hydrophilic fiber comprising sensing matter of the hygiene item according to an embodiment of the present invention. Please refer to FIG. 3 first. Another category of the present invention is to provide a hygiene item 1, wherein the hygiene item 1 can be a baby diaper, an adult diaper, or a sanitary napkin. The present invention can be a disposable hygiene item, which can be contacted with the body-fluids of the user, such as sweat, saliva, urine, menstruation, or other fluids outputted from the human body, for bio-examination. The hygiene item 1 provided by the present invention comprises a fiber layer 17, a hydrophobic fiber 18, and a hydrophilic fiber 19.

In the embodiment shown in FIG. 3, the fiber layer 17 is formed by interlacing the hydrophobic fiber 18 and the hydrophilic fiber 19, which allows the hydrophobic fiber 18 and the hydrophilic fiber 19 to be arranged interlaced on a surface of the fiber layer 17. But the method for forming the fiber layer 17 is not limited to the statement mentioned above. In the embodiment shown in FIG. 4, the fiber layer 17 can be formed by interlacing the hydrophobic fiber 18 only and utilize oxygen plasma, argon plasma, or air plasma to process a portion of the hydrophobic fiber 18 to form the hydrophilic fiber 19.

Upon the hydrophilic fiber 19 is set, dispose the sensing matter 14 on the hydrophilic fiber 19. When the matter under test 50 is contacted with the fiber layer 17, the sensing matter 14 of the hydrophilic fiber 19 will change color for bio-examination. The method for disposing the sensing matter 14 on the hydrophilic fiber 19 is shown in FIG. 3A or FIG. 3B. However, the present invention is not limited to the statement mentioned above. Under the premise that the basic spirit of the present invention is not transgressed, the user of the present invention can change the disposing method and the shape of the sensing matter 14 according to the requirement.

Wherein the sensing matter 14 comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent. In order to avoid the sensing matter 14 to contact with air for a long time and then to influence the valid period of the sensing matter 14, the sensing matter 14 can be covered by gel M to form a mixed slurry 15. In practical application, the matter under test 50 can be melted and then passes through the gel M to react with the sensing matter 14, which allows the color of the sensing matter 14 to be changed for examination. Because the hydrophobic fiber 18 and the hydrophilic fiber 19 are arranged interlaced, the two mixed slurries 15 shown in FIG. 3 and FIG. 4 can be mixed with different sensing matter 14 respectively for different types of bio-examinations.

In this embodiment, the structure mentioned above can form a sensing layer 10 first, and then combine the sensing layer 10 with a gel film 20, an absorption layer 30, and a hydrophobic layer 40 to form the hygiene item 1.

As shown in FIG. 3 and FIG. 4, in the hygiene item 1 of the present invention, the sensing layer 10 is disposed between the gel film 20 and the absorption layer 30, and the absorption layer 30 is disposed between the sensing layer 10 and the hydrophobic layer 40. The hydrophobic layer 40 is contacted with the skin of the user and adapted to receive the matter under test 50 outputted from the user. The matter under test 50 is transmitted to the sensing layer 10 through the absorption layer 30. The gel film 20 is facing to the external environment. The gel film 20 is a transparent gel film or a translucent gel film for allowing the user to observe the mixed slurry 15 directly.

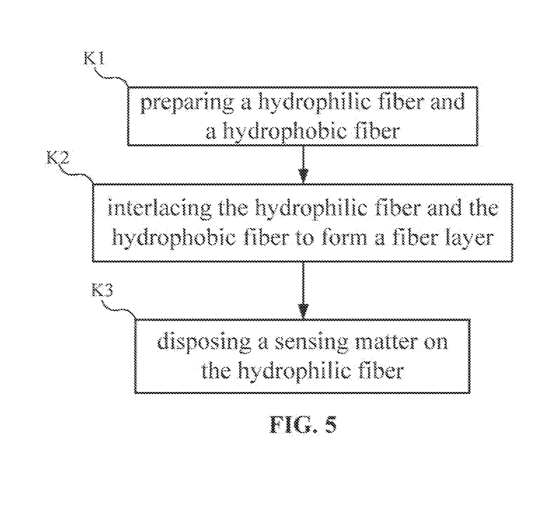

Please refer to FIG. 5. FIG. 5 shows a step flow block diagram for fabricating the hygiene item according to another embodiment of the present invention. As shown in FIG. 5, another category of the present invention is to provide a fabricating method for fiber-weaving hygiene item 1, comprising the following steps: (K1) preparing a hydrophilic fiber and a hydrophobic fiber, (K2) interlacing the hydrophilic fiber and the hydrophobic fiber to form a fiber layer, and (K3) disposing a sensing matter on the hydrophilic fiber, wherein when a matter under test is contacted with the hydrophilic fiber, the sensing matter of the hydrophilic fiber will change color for bio-examination.

Through the steps mentioned above, the sensing layer 10 can be formed first, and then the sensing layer 10 will be combined with a gel film 20, an absorption layer 30, and a hydrophobic layer 40 to form the hygiene item 1. The relative position of the sensing layer 10, the gel film 20, the absorption layer 30, and the hydrophobic layer 40 can be referred to FIG. 3, FIG. 4, and the relative statement mentioned above.

In the step (K1), prepare a hydrophobic fiber 18 and a hydrophilic fiber 19. In the step (K2), interlace the hydrophobic fiber 18 and the hydrophilic fiber 19 to form a fiber layer 17. And in the step (K3), dispose a sensing matter 14 on the hydrophilic fiber 19.

Wherein the fiber-weaving hygiene item 1 can be a baby diaper, an adult diaper, or a sanitary napkin. The present invention can be a disposable hygiene item, which can be contacted with the body-fluids of the user, such as sweat, saliva, urine, menstruation, or other kinds of fluids outputted from the human body, for bio-examination. However, the method for forming the fiber layer 17 is not limited to interlace the hydrophobic fiber 18 and the hydrophilic fiber 19 mentioned above. The fiber layer 17 also can be formed by interlacing the hydrophobic fiber 18 only, and then utilize oxygen plasma, argon plasma, or air plasma to process a portion of the hydrophobic fiber 18 to form the hydrophilic fiber 19.

Wherein, the sensing matter 14 comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent. Furthermore, in order to avoid the sensing matter 14 to contact with air for a long time and then to influence the valid period of the sensing matter 14, the sensing matter 14 can be covered by gel M to form a mixed slurry 15. In practical application, the matter under test 50 can be melted and then passes through the gel M to react with the sensing matter 14, which allows the color of the sensing matter 14 to be changed for examination.

To summarize the statement mentioned above, the present invention provides a fiber-weaving hygiene item and the fabricating method thereof, which interlaces a fabric-base fiber on the substrate (wherein the hydrophilicity of the fabric-base fiber is higher than the hydrophilicity of the substrate) through the penetrating holds on the substrate, and then disposes a sensing matter on the fabric-base fiber; or interlaces a hydrophilic fiber and a hydrophobic fiber to form the fiber layer and then disposes a sensing matter on the hydrophilic fiber, for performing a bio-examination to a matter under test.

To compare with the prior art, the hygiene item provided by the present invention distributes the hydrophilic fiber on the hydrophobic material through interlacing hydrophilic fiber and hydrophobic fiber, or interlacing the hydrophilic fiber on the hydrophobic substrate. And, the present invention disposes one sensing matter or above on the hydrophilic fiber for performing different types of bio-examinations to the matter under test, which can solve the problem of the hygiene item can not support a variety of reagents in the prior art.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A fabricating method for fiber-weaving hygiene item, comprising the following steps of:
(S1) preparing a substrate, wherein the substrate has a first surface and a corresponding second surface;
(S2) forming a plurality of penetrating holds on the substrate;
(S3) preparing a fabric-base fiber and interlacing the fabric-base fiber on the first surface and the second surface through the plurality of penetrating holds; and
(S4) disposing a sensing matter on the fabric-base fiber on the first surface;
wherein the hydrophilicity of the fabric-base fiber is higher than the hydrophilicity of the substrate, when a matter under test is transmitted through the second surface of the substrate and contacted with the first surface of the substrate, the color of the sensing matter of the fabric-base fiber will be changed for bio-examination.

2. The fabricating method for fiber-weaving hygiene item of claim 1, wherein in the step (S2), laser cutting, punching, and drilling can be utilized to form the plurality of penetrating holds.

3. The fabricating method for fiber-weaving hygiene item of claim 1, wherein between the step (S3) and (S4), the following step is comprised:
(S31) filling a waterproof sealant in the plurality of penetrating holds for fixing the fabric-base fiber on the first surface and the second surface.

4. The fabricating method for fiber-weaving hygiene item of claim 1, wherein the substrate is a hydrophobic material and the fabric-base fiber is a hydrophilic material.

5. The fabricating method for fiber-weaving hygiene item of claim 1, wherein the substrate is a gel film and the fabric-base fiber is a hydrophilic material.

6. The fabricating method for fiber-weaving hygiene item of claim 1, wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously.

7. The fabricating method for fiber-weaving hygiene item of claim 6, wherein the sensing matter can be covered by a mixed slurry to prolong the valid period of the sensing matter.

8. The fabricating method for fiber-weaving hygiene item of claim 1, wherein the matter under test is body-fluids of the human body, such as sweat, saliva, urine, menstruation, or other kinds of fluids outputted from human body.

9. The fabricating method for fiber-weaving hygiene item of claim 1, wherein the hygiene item comprises a baby diaper, an adult diaper, and a sanitary napkin.

10. A fiber-weaving hygiene item, used for performing a bio-examination to a testing matter, comprising:
a substrate, wherein the substrate has a first surface, a corresponding second surface, and a plurality of penetrating holds penetrating the substrate;
a fabric-base fiber, wherein the fabric-base fiber is interlaced on the first surface and the second surface through the plurality of penetrating holds; and
a sensing matter, wherein the sensing matter is disposed on the fabric-base fiber on the first surface;
wherein the hydrophilicity of the fabric-base fiber is higher than the hydrophilicity of the substrate, when the matter under test is transmitted through the second surface of the substrate and contacted with the first surface of the substrate, the color of the sensing matter of the fabric-base fiber will be changed for bio-examination.

11. The fiber-weaving hygiene item of claim 10 further comprising a waterproof sealant, used to be filled in the plurality of penetrating holds for fixing the fabric-base fiber on the first surface and the second surface.

12. The fiber-weaving hygiene item of claim 10, wherein the substrate is a hydrophobic material and the fabric-base fiber is a hydrophilic material.

13. The fiber-weaving hygiene item of claim 10, wherein the substrate is a gel film and the fabric-base fiber is a hydrophilic material.

14. The fiber-weaving hygiene item of claim 10, wherein the method for forming the plurality of penetrating holds on the substrate comprises laser cutting, punching, and drilling.

15. The fiber-weaving hygiene item of claim 10, wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously.

16. The fiber-weaving hygiene item of claim 15, wherein the sensing matter can be covered by a mixed slurry to prolong the valid period of the sensing matter.

17. The fiber-weaving hygiene item of claim 10, wherein the matter under test is body-fluids of the human body, such as sweat, saliva, urine, menstruation, or other kinds of fluids outputted from human bodies.

18. The fiber-weaving hygiene item of claim 10, wherein the hygiene item comprises a baby diaper, an adult diaper, and a sanitary napkin.

19. A fabricating method for fiber-weaving hygiene item, comprising the following steps of:
(K1) preparing a hydrophilic fiber and a hydrophobic fiber;
(K2) interlacing the hydrophilic fiber and the hydrophobic fiber to form a fiber layer; and
(K3) disposing a sensing matter on the hydrophilic fiber;
wherein when a matter under test is contacted with the hydrophilic fiber, the sensing matter of the hydrophilic fiber will change color for bio-examination.

20. The fabricating method for fiber-weaving hygiene item of claim 19, wherein the hydrophilic fiber also can be generated through utilizing oxygen plasma, argon plasma, or air plasma to process a portion of the hydrophobic fiber.

21. The fabricating method for fiber-weaving hygiene item of claim 19, wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously.

22. The fabricating method for fiber-weaving hygiene item of claim 21, wherein the sensing matter can be covered by a mixed slurry to prolong the valid period of the sensing matter.

23. The fabricating method for fiber-weaving hygiene item of claim 19, wherein the matter under test is body-fluids of the human body, such as sweat, saliva, urine, menstruation, or other kinds of fluids outputted from human bodies.

24. The fabricating method for fiber-weaving hygiene item of claim 19, wherein the hygiene item comprises a baby diaper, an adult diaper, and a sanitary napkin.

25. A fiber-weaving hygiene item, used for performing a bio-examination to a testing matter, comprising:
a fiber layer, wherein the fiber layer comprises a hydrophilic fiber and a hydrophobic fiber, and the hydrophilic fiber and the hydrophobic fiber are interlaced to form the fiber layer; and
a sensing matter, wherein the sensing matter is disposed on the hydrophilic fiber;
wherein when the matter under test is contacted with the fiber layer, the sensing matter of the hydrophilic fiber will change color for bio-examination.

26. The fiber-weaving hygiene item of claim 25, wherein the hydrophilic fiber also can be generated through utilizing oxygen plasma, argon plasma, or air plasma to process a portion of the hydrophobic fiber.

27. The fiber-weaving hygiene item of claim 25, wherein the sensing matter comprises a nitrite sensing reagent, a glucose sensing reagent, a bovine serum albumin (BSA) sensing reagent, a pH sensing reagent, a bilirubin sensing reagent, an urobilinogen sensing reagent, and a ketone body sensing reagent, wherein the sensing reagents can be used respectively or simultaneously.

28. The fiber-weaving hygiene item of claim 27, wherein the sensing matter can be covered by a mixed slurry to prolong the valid period of the sensing matter.

29. The fiber-weaving hygiene item of claim 25, wherein the matter under test is body-fluids of the human body, such as sweat, saliva, urine, menstruation, or other kinds of liquids outputted from human bodies.

30. The fiber-weaving hygiene item of claim 25, wherein the hygiene item comprises a baby diaper, an adult diaper, and a sanitary napkin.
